(19) United States
(12) Patent Application Publication
(10) Pub. No.: US 2026/0158196 A1
(43) Pub. Date: Jun. 11, 2026

(54) PRESSURE MEASUREMENT UNIT, DIAPHRAGM CHAMBER, AND BLOOD CIRCUIT

(71) Applicant: NIPRO CORPORATION, Settsu-shi, Osaka (JP)

(72) Inventors: Takeshi YAMAGUCHI, Settsu-shi (JP); Minami YAMAGATA KAWABE, Settsu-shi (JP)

(21) Appl. No.: 19/150,138

(22) PCT Filed: Jan. 23, 2024
(86) PCT No.: PCT/JP2024/001867

§ 371 Date: Jul. 22, 2025

(30) Foreign Application Priority Data
Jan. 23, 2023 (JP) 2023-008259

(51) Int. Cl. A61M 1/16

(52) U.S. Cl. CPC A61M 1/16; A61M 2202/0021; A61M 2202/0413; A61M 2205/3327; A61M 2205/3331; A61M 2205/3379; A61M 2205/583

(57) ABSTRACT

A pressure measurement unit includes: a drip chamber having a chamber space, a first port (inlet port), a fourth port (outlet port), and a second port (pressure measurement port); a first line having a tubular shape and having one end connected to the second port; a diaphragm chamber having a first air chamber in communication with the other end of the first line, a flexible membrane, and a second air chamber; and a second line having a tubular shape and having one end connected to the second air chamber and the other end connected to a pressure measurement device.

### TECHNICAL FIELD

The present invention relates to pressure measurement units, diaphragm chambers, and blood circuits for measuring the pressure of a liquid flowing in a liquid circuit.

### BACKGROUND ART

There are blood purification therapies in which blood of a patient is subjected to extracorporeal circulation to purify the blood during the circulation. As an example of such a therapy, dialysis therapy is known. In dialysis therapy, blood withdrawn from the patient is introduced into a dialyzer, where unnecessary components in the blood are replaced with useful components in a dialysate, filtration is also performed, and then the processed blood is reinfused to the patient. In such dialysis therapy, in order to prevent damage of useful blood cells and agglutination of the blood, the pressure of the blood is measured at various points in the blood circuit through which the blood flows.

As a device that measures the pressure of blood flowing in a blood circuit, a drip chamber for pressure monitoring disclosed in Patent Literature 1 is known. In this drip chamber, a diaphragm (4) is provided in a vertically long chamber body (2) to separate an air chamber (A) and a blood chamber (B) from each other, thereby inhibiting occurrence of blood coagulation due to contact between blood and air.

### CITATION LIST

### Patent Literature

PTL 1: JP 2005-95230A

### SUMMARY OF INVENTION

### Technical Problem

However, the drip chamber of Patent Literature 1 has a configuration in which the diaphragm is provided in the chamber body, and thus, the diaphragm inevitably comes into contact with the blood. In addition, since the diaphragm is large in size and irregularly deforms, stagnation easily occurs, which may result in clot formation. When a clot has been formed, the diaphragm does not properly operate, which may result in failure of accurate pressure measurement.

Therefore, an object of the present disclosure is to solve the above problem and provide a pressure measurement unit, a diaphragm chamber, and a blood circuit that enable accurate pressure measurement.

### Solution to Problem

A pressure measurement unit according to a first aspect of the present disclosure is a pressure measurement unit provided between a liquid circuit and a pressure measurement device, the pressure measurement unit including: a drip chamber having a chamber space, an inlet port configured to introduce a liquid flowing in the liquid circuit into the chamber space, an outlet port configured to send out the liquid in the chamber space to the liquid circuit, and a pressure measurement port in communication with an upper portion of the chamber space; a first line having a tubular shape and having one end connected to the pressure measurement port; and a diaphragm chamber having a first air chamber in communication with another end of the first line, a flexible membrane in contact with the first air chamber, and a second air chamber separated from the first air chamber by the flexible membrane.

Accordingly, in the drip chamber, the liquid is temporarily stored in the chamber space, thereby enabling air evacuation in which air in the liquid layer is allowed to move to the liquid surface due to buoyancy to be released to the gaseous layer, and entry of the liquid in the drip chamber into the pressure measurement device can be prevented by the flexible membrane of the diaphragm chamber. In addition, since the diaphragm chamber having the flexible membrane is provided separately from the drip chamber, the flexible membrane that has a desired area can be adopted without being affected by the configuration or dimensions of the drip chamber, and thus, accurate pressure measurement can be performed. Since the first line becomes a closed system space due to the diaphragm chamber, a situation where the blood comes into contact with the flexible membrane, thereby causing a clot, can be prevented.

A pressure measurement unit according to a second aspect of the present disclosure based on the first aspect may further include a second line having a tubular shape and having one end connected to the second air chamber and another end connected to the pressure measurement device, and an inner diameter of the first line may be larger than an inner diameter of the second line.

Accordingly, the internal volume of the first line can be ensured to be large. Therefore, even if the liquid flows toward the diaphragm chamber from the drip chamber through the first line, entry into the diaphragm chamber can be inhibited. Further, it is possible to expect an effect that, since the diameter of the second line is smaller than that of the first line, occurrence of a kink in the second line can be inhibited, and due to the reduced volume of the second line, the displacement width of the flexible membrane of the diaphragm chamber is widened, whereby the measurable pressure range becomes large.

In the pressure measurement unit according to a third aspect of the present disclosure based on the first aspect, the diaphragm chamber may have a housing accommodating the first air chamber, the second air chamber, and the flexible membrane, and at least a part of the housing may be transparent so as to allow an inside thereof to be visually confirmed from outside, and at least a part of the flexible membrane has a chromatic color or is semi-transparent or opaque.

Accordingly, the state or movement of the flexible membrane inside can be visually observed from outside of the diaphragm chamber, and thus, whether or not the state is a state where the pressure of the liquid can be appropriately measured can be easily confirmed.

In a pressure measurement unit according to a fourth aspect of the present disclosure based on the first aspect, the diaphragm chamber may have a first air chamber port in communication with the first air chamber, and a direction of deformation of the flexible membrane due to differential pressure change between the first air chamber and the second air chamber and a flowing direction of a fluid in the first air chamber port may cross each other.

Accordingly, in a situation where the flexible membrane is not positioned on the extension of the opening direction of the first air chamber port, the fluid (air) having flowed in from the first air chamber port is prevented from directly colliding with the flexible membrane. In this case, pulsation of the flexible membrane when the fluid has forcefully flowed in is inhibited. Therefore, it is possible to expect an effect that the measurement value of the pressure is easily stabilized as compared with a case where the opening direction of the first air chamber port matches the direction of deformation of the flexible membrane.

In a pressure measurement unit according to a fifth aspect of the present disclosure based on the first aspect, the diaphragm chamber may have a housing accommodating the first air chamber, the second air chamber, and the flexible membrane, and a first air chamber port formed at the housing and configured to allow communication between the first air chamber and the first line, and the first air chamber may be defined by an inner face of the housing and the flexible membrane, and at the inner face of the housing, a recess or a protrusion forming a space that allows communication between the first air chamber and the first air chamber port may be formed.

Accordingly, even when the flexible membrane has largely been deflected to the first air chamber side due to action of a negative pressure onto the first air chamber, a space is ensured by the recess or the protrusion. Therefore, occurrence of a situation where the opening on the first air chamber side of the first air chamber port is closed by the flexible membrane, resulting in significant decrease in the assumed pressure measurement range, can be inhibited.

In a pressure measurement unit according to a sixth aspect of the present disclosure based on the fifth aspect, the space may extend in a direction away from the first air chamber port.

Accordingly, it is possible to prevent formation of an independent space (bubble) not in communication with the first air chamber port in the first air chamber caused by the flexible membrane irregularly deforming and adhering to the inner face of the housing. If such an independent space is formed, the deformable amount of the flexible membrane decreases by an amount corresponding to the independent space not in communication with the first air chamber port, and thus, the range in which the pressure is measurable is reduced. However, according to the present disclosure, a space extending in the direction away from the first air chamber port is present. Thus, even if the flexible membrane has irregularly deformed to adhere to the inner face of the housing in such a manner as to include a bubble, occurrence of a situation where this bubble becomes an independent space not in communication with the first air chamber port can be reduced, and thus, reduction in the pressure measurement range can be prevented. In a state where the flexible membrane has deformed such that the volume of the first air chamber becomes minimum in a natural state where elastic force is not generated, the above space increases the area in which the fluid can be in contact with the flexible membrane. Therefore, the fluid (air) having flowed in from the first air chamber port flows through this space, whereby the force with which the fluid having flowed in directly collides with the downwardly displaced flexible membrane can be mitigated.

In a pressure measurement unit according to a seventh aspect of the present disclosure based on the first aspect, when a maximum change amount of a volume of the first air chamber caused by displacement of the flexible membrane in the diaphragm chamber is defined as V1, and of a volume of the chamber space of the drip chamber, a volume from a liquid level setting line to a top face is defined as V2, 1.2×V1≤V2≤3.5×V1 may be satisfied.

Accordingly, while high pressure measurement accuracy is ensured, downsizing of the diaphragm chamber can be realized. That is, with respect to the diaphragm chamber, the larger the above V1 is, the larger the pressure measurement range becomes, but from the viewpoint of ease of handling the product on site, downsizing of the diaphragm chamber is desired. Here, when a roller pump is used for transportation of a liquid in a liquid circuit, pulsation is generated also in the air in an upper space of a drip chamber due to a pressure difference caused during discharge of the liquid and during suction of the liquid. However, in the case of the pressure measurement unit according to the present disclosure, the diaphragm chamber is connected to the drip chamber and the diaphragm chamber is configured such that the air comes into contact with the flexible membrane. Therefore, the volume of the space connected to the upper space of the drip chamber is large, and thus, displacement of the flexible membrane due to pulsation can be made small. Accordingly, influence of pulsation on pressure measurement can be suppressed. Thus, the diaphragm chamber can be downsized, and dimensions that satisfy the above formula can be realized.

In a pressure measurement unit according to an eighth aspect of the present disclosure based on the first aspect, between the first line and a housing of the diaphragm chamber, a joint tube that is more flexible than the housing of the diaphragm chamber may be provided.

Accordingly, occurrence of a kink when the first line has been bent can be inhibited, and thus, degree of freedom of arrangement of the first line is improved.

A pressure measurement unit according to a ninth aspect of the present disclosure is a pressure measurement unit provided between a liquid circuit and a pressure measurement device, the pressure measurement unit including: a first line having a tubular shape and branched from the liquid circuit; and a diaphragm chamber having a first air chamber in communication with the first line, a flexible membrane in contact with the first air chamber, and a second air chamber separated from the first air chamber by the flexible membrane, wherein between the first line and a housing of the diaphragm chamber, a joint tube that is more flexible than the housing of the diaphragm chamber is provided.

Thus, since the flexible membrane is provided to the diaphragm chamber provided via the first line branched from the liquid circuit, it is possible to prevent the liquid flowing in the liquid circuit from coming into contact with the flexible membrane. Therefore, when blood flows as the liquid, contact between the blood and the flexible membrane can be prevented, whereby formation of a clot can be avoided. Since the joint tube is included, occurrence of a kink when the first line has been bent can be inhibited, and thus, degree of freedom of arrangement of the first line is improved. The branching form between the liquid circuit and the first line is not particularly limited. For example, the liquid circuit and the first line may be connected via a three-way-shaped joint or may be connected via a drip chamber.

A diaphragm chamber according to a tenth aspect of the present disclosure is a diaphragm chamber, for a pressure measurement unit, provided between a liquid circuit and a pressure measurement device and connected to a first line branched from the liquid circuit, the diaphragm chamber including: a first air chamber in communication with the first line; a flexible membrane in contact with the first air chamber; a second air chamber separated from the first air chamber by the flexible membrane; and a first air chamber port in communication with the first air chamber, wherein a direction of deformation of the flexible membrane due to differential pressure change between the first air chamber and the second air chamber and a flowing direction of a fluid in the first air chamber port cross each other.

Thus, since the flexible membrane is provided to the diaphragm chamber provided via the first line branched from the liquid circuit, it is possible to prevent the liquid flowing in the liquid circuit from coming into contact with the flexible membrane. Therefore, when blood flows as the liquid, contact between the blood and the flexible membrane can be prevented, whereby formation of a clot can be avoided. In addition, when the flexible membrane is not positioned on the extension of the opening direction of the first air chamber port, the fluid (air) having flowed in from the first air chamber port is prevented from directly colliding with the flexible membrane. In this case, pulsation of the flexible membrane when the fluid has forcefully flowed in is inhibited, and thus, it is possible to expect an effect that the measurement value of the pressure is easily stabilized. The branching form between the liquid circuit and the first line is not particularly limited. For example, the liquid circuit and the first line may be connected via a three-way-shaped joint or may be connected via a drip chamber.

A blood circuit according to an eleventh aspect of the present disclosure is a blood circuit having a pumping tube fixed to a roller pump at a point on a blood line, the blood circuit including: a branch line having one end connected at a position on an upstream side of the pumping tube on the blood line; and a diaphragm chamber connected to another end of the branch line and having a first air chamber in communication with the branch line, a flexible membrane in contact with the first air chamber, and a second air chamber separated from the first air chamber by the flexible membrane, wherein a length of the branch line is not less than 100 mm.

When the branch line is positioned on the upstream side of the pumping tube, due to influence of pulsation caused in association with drive of the pumping tube, air in the branch line moves to the blood line side, and in exchange for this air, the blood in the blood line can flow into the branch line. In the blood circuit according to the present disclosure, since the length of the branch line is set to be not less than 100 mm, it is possible to inhibit the blood having flowed into the branch line from reaching the diaphragm chamber and forming a clot.

### Advantageous Effects of Invention

According to the present disclosure, a pressure measurement unit, a diaphragm chamber, and a blood circuit that enable accurate pressure measurement can be realized.

### BRIEF DESCRIPTION OF DRAWINGS

FIG. 1 is a schematic diagram showing a configuration of a part of a dialysis system in which a pressure measurement unit according to the present embodiment is adopted.

FIG. 2 is a perspective view showing a configuration of the pressure measurement unit.

FIG. 3 is a front view showing a configuration of a drip chamber.

FIG. 4 is a perspective view showing a configuration of a diaphragm chamber.

FIG. 5A is a cross-sectional view showing a configuration of the diaphragm chamber.

FIG. 5B is a plan view showing a configuration of the diaphragm chamber.

FIG. 6 is a perspective view showing another configuration of the pressure measurement unit.

FIG. 7 is a schematic diagram showing another configuration of a blood circuit according to the present embodiment.

### DESCRIPTION OF EMBODIMENTS

Hereinafter, a pressure measurement unit and a diaphragm chamber according to an embodiment of the present disclosure will be described with reference to the drawings. The concept of directions used in the following description is used for convenience of description, and does not limit the orientation and the like of each disclosed configuration to the direction. The pressure measurement unit described below is merely an embodiment of the present disclosure. Therefore, the present disclosure is not limited to the embodiment below, and additions, deletions, or modifications can be made to the configuration without departing from the gist of the disclosure.

### Overall Configuration of Blood Circuit

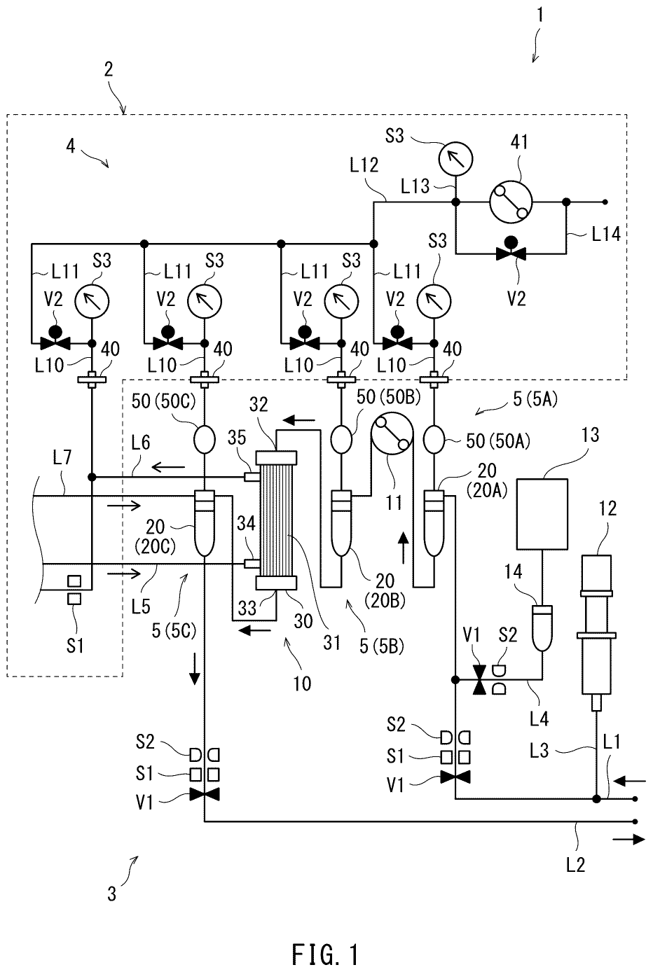

FIG. 1 is a schematic diagram showing a configuration of a part of a dialysis system 1 in which a pressure measurement unit according to the present embodiment is adopted. As shown in FIG. 1, this dialysis system 1 includes a dialysis device 2 and a blood circuit (liquid circuit) 3, the dialysis device 2 has a pressure measurement device 4, and between the blood circuit 3 and the pressure measurement device 4, a pressure measurement unit 5 (5A to 5C) according to the present embodiment is provided.

The blood circuit 3 includes a blood withdrawal line L1, a filtration device 10, and a blood reinfusion line L2. The blood withdrawal line L1 has a flexible tube, and connects a blood vessel of a patient and the filtration device 10 to each other. At a point on the blood withdrawal line L1, a blood pump 11 is provided, and when this blood pump 11 operates, blood of the patient is sent through the blood withdrawal line L1 to the filtration device 10.

On the blood withdrawal line L1, one end of a drug line L3 is connected to an upstream-side portion (patient-side portion) with respect to the blood pump 11, and the other end of the drug line L3 is connected to a syringe pump 12. A drug such as an anticoagulant agent and the like is in the syringe pump 12, and the drug can be injected, through the drug line L3, to the blood passing through the blood withdrawal line L1.

On the blood withdrawal line L1, one end of a saline line L4 is connected to a downstream-side portion with respect to the connection point with the drug line L3, and the other end of the saline line L4 is connected to a saline bag 13. A saline is in the saline bag 13, and the saline can be supplied to the blood withdrawal line L1 at the time of priming. At a point on the saline line L4, a drip chamber 14 is provided, whereby entry of air into the blood withdrawal line L1 during supply of the saline is prevented.

On the blood withdrawal line L1, between the connection point with the drug line L3 and the connection point with the saline line L4, a valve V1, a component detection sensor S1, and an air detection sensor S2 are provided. The valve V1 is a gate valve, for example, and can switch the blood withdrawal line L1 between “fully opened” and “fully closed”. As the component detection sensor S1, a known sensor that can detect components of the blood flowing in the blood withdrawal line L1 is used. The air detection sensor S2 is an ultrasonic sensor, for example, and a known sensor that can detect entry of air in the blood flowing in the blood withdrawal line L1 is used. In the example in FIG. 1, the valve V1, the component detection sensor S1, and the air detection sensor S2 are disposed in this order from the upstream side to the downstream side of the blood withdrawal line L1.

A valve V1 and an air detection sensor S2 are provided also on the downstream side of the drip chamber 14 on the saline line L4. In the example in FIG. 1, on the saline line L4, the air detection sensor S2 is disposed on the upstream side (upstream side in the direction in which the saline flows) of the valve V1.

On the blood withdrawal line L1, a drip chamber 20 (20A) is provided on the downstream side of the connection point with the saline line L4. This drip chamber 20A forms a pressure measurement unit 5 (5A). The pressure measurement unit 5 will be described later.

On the blood withdrawal line L1, the blood pump 11 is provided on the downstream side of the drip chamber 20A. The blood pump 11 is a tube-type roller pump, for example, and squeezes the flexible tube forming the blood withdrawal line L1 with a rotating roller, to control the flow of the blood in the tube.

On the blood withdrawal line L1, a drip chamber 20 (20B) is provided on the downstream side of the blood pump 11. This drip chamber 20B forms a pressure measurement unit 5 (5B).

The filtration device 10 is connected to the downstream end, i.e., an end portion on the downstream side of the drip chamber 20B, of the blood withdrawal line L1. The filtration device 10 includes a case 30 having a cylindrical shape, and a filter 31 accommodated in this case 30. The filter 31 is formed as a bundle of a plurality of hollow fibers, for example. The case 30 has: a blood inlet port 32 provided at one end portion; a blood outlet port 33 provided at the other end portion; a dialysate inlet port 34 provided at a periphery in the vicinity of the other end portion; and a dialysate outlet port 35 provided at a periphery in the vicinity of the one end portion.

The downstream end of the blood withdrawal line L1 described above is connected to the blood inlet port 32. Meanwhile, the upstream end of the blood reinfusion line L2 is connected to the blood outlet port 33. Therefore, the blood sent to the filtration device 10 through the blood withdrawal line L1 enters the case 30 through the blood inlet port 32, and then enters through one-end openings of the hollow fibers forming the filter 31, to flow through the inside of the hollow fibers. Then, the blood exits through the other-end openings of the hollow fibers to be sent out to the blood reinfusion line L2 through the blood outlet port 33.

The blood reinfusion line L2 has a flexible tube and connects the filtration device 10 and the blood vessel of patient to each other. At a point on the blood reinfusion line L2, a drip chamber 20 (20C) is provided. This drip chamber 20C forms a pressure measurement unit 5 (5C).

On the downstream side of the drip chamber 20C on the blood reinfusion line L2, a valve V1, a component detection sensor S1, and an air detection sensor S2 are disposed in this order from the downstream side to the upstream side. Therefore, the blood reinfusion line L2 can be switched between “fully opened” and “fully closed” by the valve V1, components of the blood flowing in the blood reinfusion line L2 can be detected by the component detection sensor S1, and entry of air in the blood flowing in the blood reinfusion line L2 can be detected by the air detection sensor S2.

A dialysate line L5 is connected to the dialysate inlet port 34 of the filtration device 10, and a collection line L6 is connected to the dialysate outlet port 35. The dialysate accommodated in a bag flows in the dialysate line L5, is caused to have, during flowing, an appropriate temperature through a heater included in the dialysis device 2, and then, is supplied to the filtration device 10 through the dialysate inlet port 34. In the filtration device 10, a specific component in the blood is transferred to the dialysate through the filter 31. Then, the dialysate including the specific component passes through the dialysate outlet port 35 and the collection line L6, to be collected into a collection container such as a tank or the like.

One end of a replacement fluid line L7 is connected to the drip chamber 20C. The other end of the replacement fluid line L7 is connected to a replacement fluid bag, and a replacement fluid is supplied to the drip chamber 20C through the replacement fluid line L7. Therefore, the blood having passed through the filtration device 10 has the replacement fluid mixed thereinto at the drip chamber 20C, and is returned to the patient through the blood reinfusion line L2. Thus, the drip chamber 20C also functions as a mixing chamber.

Meanwhile, the dialysis device 2 includes the pressure measurement device 4. The pressure measurement device 4 is connected to the blood circuit 3 described above via the pressure measurement unit 5. This pressure measurement device 4 has a plurality of connection ports 40 to each of which the pressure measurement unit 5 is connected, each connection port 40 has connected thereto one end of a first monitoring line L10 extending in the device, and the other end of the first monitoring line L10 is connected to a pressure sensor S3. The first monitoring line L10 is composed of a hollow tube. The pressure sensor S3 is a semiconductor sensor or the like, for example, converts a result of measurement of the pressure in the first monitoring line L10 into an electric signal, and outputs the resultant signal to a control device or the like.

At a point on each first monitoring line L10, one end of a branch line L11 is connected, and the branch lines L11 are disposed in parallel with each other. The other end of each branch line L11 is connected to one end of a single gathering line L12, and the other end of the gathering line L12 is open to the atmosphere. At a point on the gathering line L12, a tube pump 41 is provided, and accordingly, the flow rate of air flowing through the inside of the gathering line L12 is controlled. At a point on the gathering line L12, one end of a second monitoring line L13 is connected, and the other end of the second monitoring line L13 is connected to a pressure sensor S3.

At a point on each first monitoring line L10, a valve V2 is provided. The valve V2 is composed of an air-operated valve, for example, whereby the flow of air in the first monitoring line L10 is controlled. The gathering line L12 is provided with a bypass line L14 so as to be in parallel with the tube pump 41. At a point in this bypass line L14 as well, a valve V2 is provided, whereby the flow of air in the bypass line L14 can be controlled.

### Pressure Measurement Unit

The pressure measurement units 5A to 5C described above basically have an identical configuration, and in the present embodiment, these are collectively referred to as a pressure measurement unit 5. As shown in FIG. 1, the pressure measurement unit 5A has the drip chamber 20A and a diaphragm chamber 50A, the pressure measurement unit 5B has the drip chamber 20B and a diaphragm chamber 50B, and the pressure measurement unit 5C has the drip chamber 20C and a diaphragm chamber 50C. In the present embodiment, the drip chambers 20A to 20C are collectively referred to as a drip chamber 20, and the diaphragm chambers 50A to 50C are collectively referred to as a diaphragm chamber 50.

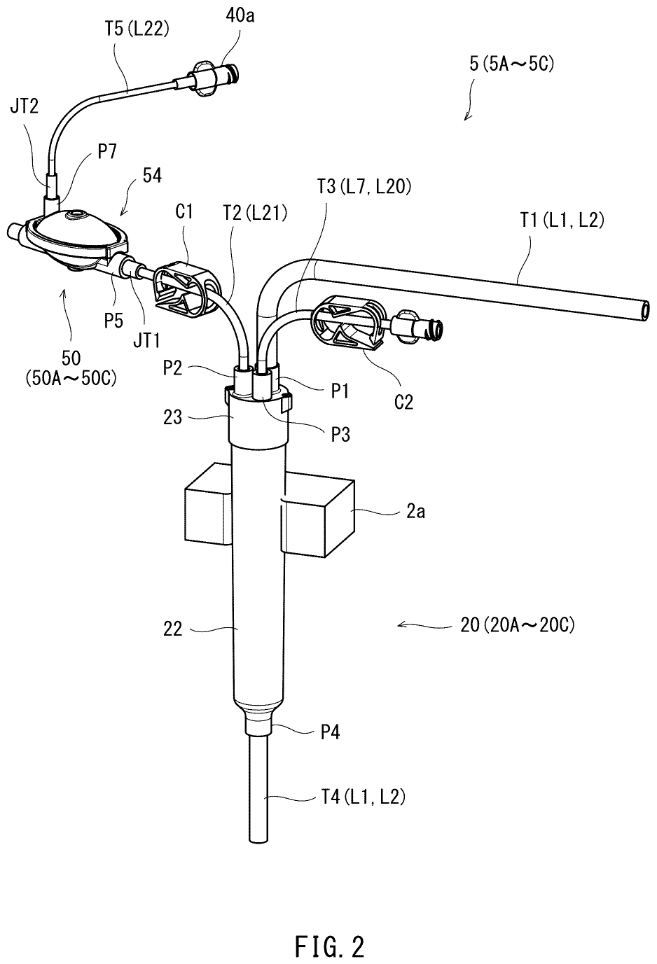

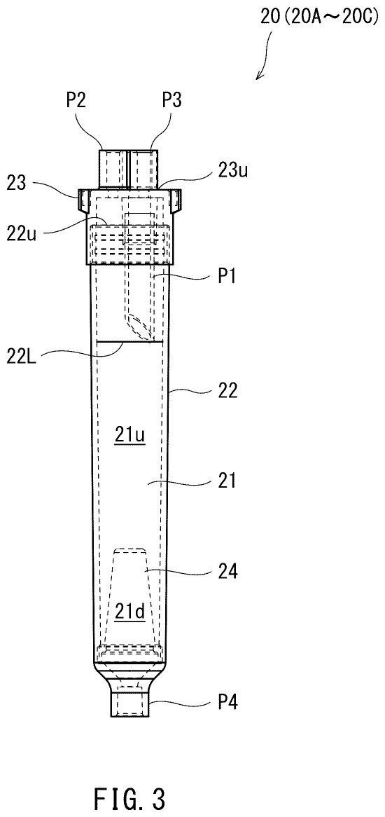

The drip chamber 20 in the pressure measurement unit 5 will be described with reference to FIG. 2 and FIG. 3. The drip chamber 20 includes a cylindrical chamber body 22 having a chamber space 21 therein. One end portion (upper end portion) 22u of the chamber body 22 is open, and a cap 23 having a cylindrical shape is externally fitted on the upper end portion 22u so as to close this opening. The cap 23 has an upper end portion 23u that is closed, and this upper end portion 23u is provided with a first port (inlet port) P1, a second port (pressure measurement port) P2, and a third port P3.

The first port P1 is composed of a tubular member having a cylindrical shape, extends upward from the upper end portion 23u of the cap 23, and also extends downward from the upper end portion 23u, that is, into the chamber space 21. Therefore, the inside and the outside of the chamber body 22 are in communication with each other through the first port P1. An upper end portion of this first port P1, that is, an end portion on the outer side of the chamber body 22, has connected thereto a tube T1 of various types through which a fluid flows. For example, in the configuration in FIG. 1, in the case of the pressure measurement unit 5A, 5B, the tube T1 is the blood withdrawal line L1, and in the case of the pressure measurement unit 5C, the tube T1 is the blood reinfusion line L2. In each case, the flowing fluid is blood. Thus, the first port Pl forms an “inlet port” that introduces the blood flowing in the blood circuit 3 into the chamber space 21. The open end face in the chamber space 21 of the first port P1 is a face that is inclined with respect to both of the flowing direction of the fluid in the first port P1 and the direction orthogonal to the flowing direction.

Including the above first port P1, each port (the first port P1, the second port P2, the third port P3, a fourth port P4, first air chamber ports P5, P6, a second air chamber port P7) of the drip chamber 20 and the diaphragm chamber 50 has a tubular configuration as an example, but the configuration of each port is not limited thereto. For example, some or all of the ports may be configured merely as an opening or may adopt another shape.

The second port P2 is composed of a tubular member having a cylindrical shape, extends upward from the upper end portion 23u of the cap 23, and, different from the first port P1, does not extend into the chamber space 21. That is, the opening in the chamber space 21 of the second port P2 is positioned above with respect to the opening in the chamber space 21 of the first port P1. This second port P2 allows communication between the inside and the outside of the chamber body 22 through a through-hole formed in the upper end portion 23u of the cap 23. An upper end portion of the second port P2 has connected thereto a tube T2 of various types through which a fluid flows. For example, in the configuration in FIG. 1, in each case of the pressure measurement units 5A to 5C, the tube T2 is a first line L21 (see FIG. 2) having a tubular shape. That is, one end of the first line L21 is connected to the second port P2, and the other end of the first line L21 is connected to the diaphragm chamber 50 described later. Therefore, the first line L21 is a line branched from the blood circuit 3 (specifically, the blood withdrawal line L1 or the blood reinfusion line L2) via the drip chamber 20 and connected to the diaphragm chamber 50. The fluid that flows in this first line L21 is air. At a point on this first line L21, a clamp C1 that restricts and allows the flow of the fluid inside the first line L21 is provided. Thus, the second port P2 forms a “pressure measurement port” that is in communication with an upper portion of the chamber space 21.

The third port P3 is composed of a tubular member having a cylindrical shape, and extends upward from the upper end portion 23u of the cap 23. This third port P3 allows communication between the inside and the outside of the chamber body 22 through a through-hole formed in the upper end portion 23u of the cap 23. An upper end portion of the third port P3 has connected thereto a tube T3 of various types through which a fluid flows. For example, in the configuration in FIG. 1, in the case of the pressure measurement unit 5A, 5B, the tube T3 is a liquid level adjustment line L20 having a tubular shape, and the flowing fluid is air. That is, one end of the liquid level adjustment line L20 is connected to the third port P3, the other end of the liquid level adjustment line L20 is open to the atmosphere, and further, at a point on the liquid level adjustment line L20, a clamp C2 that restricts and allows the flow of the fluid inside the liquid level adjustment line L20 is provided.

In the configuration in FIG. 1, in the case of the pressure measurement unit 5C, the tube T3 is the replacement fluid line L7, and the flowing fluid is a replacement fluid such as a dialysate or the like. That is, one end of the replacement fluid line L7 is connected to the third port P3, and the other end of the replacement fluid line L7 is connected to a replacement fluid bag. Therefore, from the replacement fluid bag, the replacement fluid of which the flow rate has been adjusted by a finger pump or the like, for example, is supplied through the third port P3 to the drip chamber 20C through the replacement fluid line L7.

Meanwhile, the other end portion (lower end portion) 22d of the chamber body 22 has its diameter reduced, and forms a fourth port (outlet port) P4. The fourth port P4 has a cylindrical shape (tubular shape) and extends downward. A lower end portion of this fourth port P4 has connected thereto a tube T4 of various types through which a fluid flows. For example, in the configuration in FIG. 1, in the case of the pressure measurement unit 5A, 5B, the tube T4 is the blood withdrawal line L1, and in the case of the pressure measurement unit 5C, the tube T4 is the blood reinfusion line L2. In each case, the flowing fluid is blood. Thus, the fourth port P4 forms an “outlet port” that sends out the blood in the chamber space 21 to the blood circuit 3.

A filter 24 is provided in the chamber space 21. The filter 24 is provided in a lower portion near the fourth port P4 in the chamber space 21, and partitions the chamber space 21 into a lower space 21d having a small capacity including the fourth port P4 and the other upper space 21u having a large capacity. This filter 24 is composed of a resin-molded body having small holes, a mesh body obtained by weaving fibers, or the like, and removes foreign matter such as blood clots and the like while allowing the blood to flow from the upper space 21u to the lower space 21d.

Such a drip chamber 20 is supported so as to be attachable to and detachable from the housing of the dialysis device 2. For example, the chamber body 22 is gripped by a supporting tool 2a having a clip shape provided at the outer face of the housing of the dialysis device 2, thereby being fixedly supported in such a posture that the fourth port P4 is positioned below with respect to the cap 23.

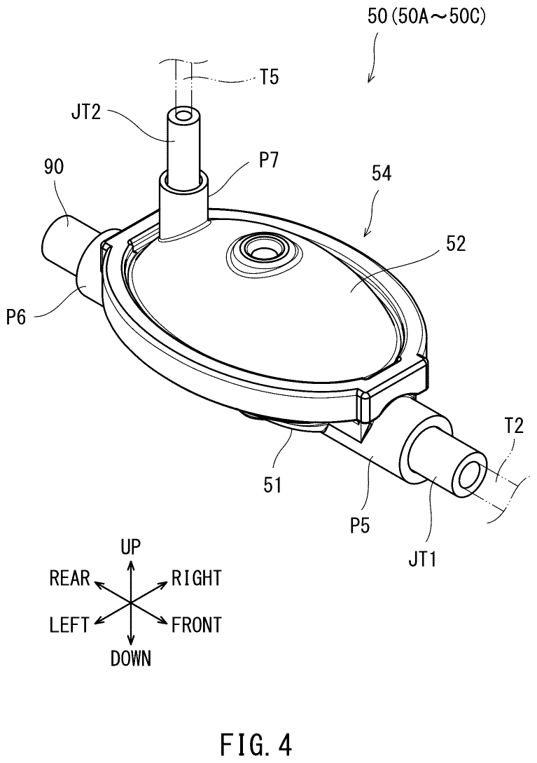

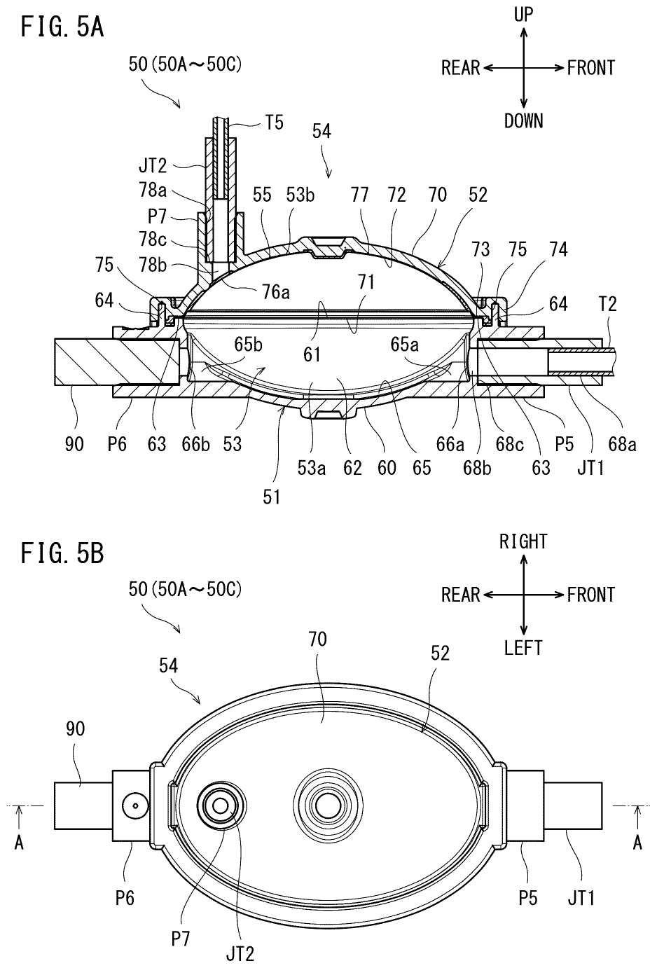

Next, the diaphragm chamber 50 of the pressure measurement unit 5 will be described with reference to FIG. 2, FIG. 4, and FIG. 5 (FIG. 5A, FIG. 5B). The diaphragm chamber 50 has a housing 54 forming a chamber space 53 obtained by putting a first case 51 and a second case 52 together. A flexible membrane 55 is provided in the housing 54, and the chamber space 53 is partitioned by the flexible membrane (diaphragm membrane) 55 into a first air chamber 53a on the first case 51 side and a second air chamber 53b on the second case 52 side. That is, the first air chamber 53a is in contact with the flexible membrane 55 and is hermetically separated from the second air chamber 53b by the flexible membrane 55. In addition, in the first case 51, first air chamber ports P5, P6 are formed, and in the second case 52, a second air chamber port P7 is formed at a position, away from the center, on the side opposite to the side where the first air chamber port P5 is present.

This diaphragm chamber 50 has a generally oval-shaped outer shape, and the posture of the diaphragm chamber 50 during use is not particularly limited. However, in the following, for convenience of description, directions with respect to the diaphragm chamber 50 are defined as follows. That is, the direction in which the second case 52 is positioned with respect to the first case 51 is defined as upward, and the direction opposite thereto is defined as downward. The direction in which the second air chamber port P6 is positioned with respect to the first air chamber port P5 is defined as frontward, and the direction opposite thereto is defined as rearward. Further, the direction crossing both of the up-down direction and the front-rear direction is defined as the left-right direction, with reference to the orientation when facing frontward.

In the present embodiment, the diaphragm chamber 50 having an oval shape as mentioned above will be described as an example, but the configuration of the diaphragm chamber 50 is not limited thereto. For example, a diaphragm chamber having a cylindrical casing that is provided with a blood flow-in port at one end, a blood flow-out port at the other end, and a pressure measurement port at a peripheral wall, and in which a cylindrical flexible membrane is disposed, is also applicable. As for the outer shape, a dome-shaped casing having a circular shape in a plan view may be adopted, or another shape may be adopted, and the mounting positions of various types of ports to the casing are not particularly limited, either.

The first case 51 includes a case body 60, and the first air chamber ports P5, P6 are connected to this case body 60. The case body 60 has an opening portion 61 that is open upward, and an inner face 62 is recessed downward. This inner face 62 and the lower face of the flexible membrane 55 form the first air chamber 53a, which is the space on the lower side of the chamber space 53. The shape (i.e., the shape of the opening portion 61) of the chamber space 53 when viewed in a plan view along the up-down direction is an elliptical shape having a major axis extending in the front-rear direction and a minor axis extending in the left-right direction. The shape of the chamber space 53 when viewed in a front view along the front-rear direction is a perfect circle shape.

At the opening portion 61 of the case body 60, a circumferential face 63 extending in the diameter-enlarging direction from an upper end portion of the inner face 62 and encircling the opening portion 61 is formed. On the outer side of this circumferential face 63, a circumferential wall 64 extending upward so as to surround the outer side of the circumferential face 63 stands. As described later, among these, the circumferential face 63 supports the flexible membrane 55, and the circumferential wall 64 engages with the second case 52.

One first air chamber port P5 is provided integrally with a front portion of the case body 60. The first air chamber port P5 has a round tubular shape, and the flow path therein is in communication with the first air chamber 53a in the case body 60. The other first air chamber port P6 is provided integrally with a rear portion of the case body 60. The first air chamber port P6 has a round tubular shape, and the flow path therein is also in communication with the first air chamber 53a. The flow paths of these two first air chamber ports P5, P6 are disposed coaxially with each other, with their center lines oriented in the front-rear direction.

At a bottom portion of the inner face 62 of the case body 60, a groove 65 extending along the front-rear direction and recessed with respect to the inner face 62 is formed. This groove 65 extends from the connection point of the first air chamber port P5 to the case body 60, to the connection point of the first air chamber port P6 to the case body 60. Therefore, between the inner face 62 and an opening 66a on the side in communication with the first air chamber 53a at the first air chamber port P5, a space 65a forming a part on the front side of the groove 65 is present. That is, the opening 66a of the first air chamber port P5 is recessed from the inner face 62 of the case body 60 by an amount corresponding to the space 65a. Similarly, between the inner face 62 and an opening 66b on the side in communication with the first air chamber 53a at another first air chamber port P6, a space 65b formed by the groove 65 is present.

Due to such a groove 65, for example, even when the internal pressure of the first air chamber 53a has decreased and the later-described flexible membrane 55 has largely been deflected downward, the opening 66a of the first air chamber port P5 is not closed by the flexible membrane 55. The pressure transmitted in the case body 60 through the first air chamber port P5 acts on a wide region of the flexible membrane 55 via the space (the space that allows communication between the first air chamber port P5 and the first air chamber 53a) such as the spaces 65a, 65b formed by the groove 65. Therefore, also in such a situation, the diaphragm chamber 50 enables accurate pressure measurement.

As seen from FIG. 5A, of the groove 65, the space 65a on the front side and the space 65b on the rear side are largely recessed from the inner face 62 of the first case 51 with respect to the other portion of the groove 65. Therefore, even when the flexible membrane 55 has largely been deflected to an extent of coming into contact with the inner face 62, at least the space 65a can be ensured as a space for pressure measurement. In addition, water vapor generated from the liquid surface of the liquid stored in the drip chamber 20 may enter the first air chamber 53a of the diaphragm chamber 50. In the diaphragm chamber 50, when the temperature of the housing 54 exposed to the outside has become lower than the internal temperature of the housing 54, there is a possibility that water vapor that has entered condenses at the inner face of the housing 54, thereby generating water droplets. Since the flexible membrane 55 is a thin member, permeation of water vapor cannot be completely prevented at all times, and a minute amount of water vapor may be allowed to permeate from the first air chamber 53a to the second air chamber 53b side. If there are the spaces 65a, 65b having a large capacity at end portions of the groove 65, water droplets can be retained in the spaces 65a, 65b, and thus, occurrence of a state where water droplets are attached to the flexible membrane 55 can be reduced, and a situation where water vapor permeates the flexible membrane 55 can be inhibited.

Even if, due to the space formed by the groove 65, the flexible membrane 55 has been deflected in a wavelike manner and the first air chamber 53a is divided into a plurality of spaces, the divided spaces can be in communication with each other through the groove 65. Accordingly, the space in communication with the first air chamber port P5 becomes large, and the contact area between the space and the flexible membrane 55 is enlarged, and thus, a wide pressure measurement range can be ensured. Here, in order to cause a plurality of spaces in the first air chamber 53a to be in communication with each other like this, it is preferable that the space formed by the groove 65 is configured to extend in a direction away from the first air chamber port P5. Preferably, the space formed by the groove 65 has a length not less than ½ of the dimension in the front-rear direction of the inner face 62 of the first case 51. However, the configuration is not limited to a configuration of extending rearward from the first air chamber port P5 in a straight-line manner.

The configuration for exhibiting the above effect is not limited to the groove 65. For example, instead of or in addition to the groove 65, a protruding rib extending in the front-rear direction may be formed on the inner face 62. Alternatively, instead of or in addition to the groove 65, a plurality of protrusions may be formed on the inner face 62. That is, since it is sufficient that the contact area between the flexible membrane 55 and the inner face 62 (particularly, the peripheral portion of the opening 66a) of the case body 60 can be reduced, a recess or a protrusion may be provided to the inner face 62 so as to form a space that allows communication between the first air chamber 53a and the first air chamber port P5.

The second case 52 includes a case body 70, and the second air chamber port P7 is connected to this case body 70. The case body 70 has a generally domed shape and has an opening portion 71 that is open downward, and an inner face 72 is recessed upward. This inner face 72 and the upper face of the flexible membrane 55 form the second air chamber 53b, which is the space on the upper side of the chamber space 53.

At the opening portion 71 of the case body 70, a circumferential face 73 extending in the diameter-enlarging direction from a lower end portion of the inner face 72 and encircling the opening portion 71 is formed. On the outer side of this circumferential face 73, a circumferential wall 74 extending downward so as to surround the outer side of the circumferential face 73 is provided in a hanging manner. Further, in the circumferential face 73, inward of the above circumferential wall 74, a circumferential groove 75 recessed upward is provided so as to encircle the opening portion 71 along the circumferential face 73. As described later, among these, the circumferential face 73 supports the flexible membrane 55, and the circumferential groove 75 engages with the first case 51.

The second air chamber port P7 is provided integrally with a rear portion of the case body 70. The second air chamber port P7 has a round tubular shape, and extends, with the center line thereof oriented in the up-down direction, upward from the outer face of the case body 70. The internal passage of the second air chamber port P7 is in communication with the second air chamber 53b in the case body 70, through an opening 76a at the lower end of the second air chamber port P7. On the inner face 72 of the case body 70, in order to form an air flow path between the inner face 72 and the flexible membrane 55, a plurality of ribs 77 having a linear shape, for example, are formed. Instead of the ribs, a plurality of protrusions or grooves having a linear shape may be formed. With this, generation of independent bubbles not in communication with the second air chamber port P7 can be reduced.

The flexible membrane 55 is a diaphragm membrane having a moderate flexibility, and is displaced in the up-down direction toward the first air chamber 53a or the second air chamber 53b in accordance with the differential pressure (the difference between the internal pressures) between the first air chamber 53a and the second air chamber 53b. That is, the direction of deformation (the up-down direction) of the flexible membrane 55 due to differential pressure change between the first air chamber 53a and the second air chamber 53b and the flowing direction (the front-rear direction) of the fluid in the first air chamber port P5 cross each other. The flexible membrane 55 has a generally domed shape, similar to (the inner face 72 of) the case body 70 of the second case 52, and has a membrane body 80 and a flange 81. The membrane body 80 is recessed upward so as to be open downward, and in a plan view, has an elliptical shape having a major axis extending in the front-rear direction and having a minor axis extending in the left-right direction. The flange 81 is provided so as to extend in the diameter-enlarging direction from a lower end portion of the membrane body 80 and encircle the opening portion.

The housing 54 composed of the first case 51 and the second case 52 as above, and the flexible membrane 55 are combined together to form the diaphragm chamber 50. That is, firstly, to the first case 51 with the opening portion 61 oriented upward, the flexible membrane 55 in a posture (a posture of being open downward) recessed upward is assembled from above. At this time, the lower face of the flange 81 of the flexible membrane 55 is disposed so as to oppose the circumferential face 63 of the first case 51. Next, to the first case 51 in this state, the second case 52 is assembled from above, with the opening portion 71 oriented downward. At this time, the circumferential wall 74 of the second case 52 is externally fitted to the circumferential wall 64 of the first case 51. In addition, the circumferential face 73 of the second case 52 is disposed so as to oppose the upper face of the flange 81 of the flexible membrane 55.

Then, the first case 51 and the second case 52 are adhered or welded together at peripheral portions thereof, and the flexible membrane 55 is supported in a state of being sandwiched between the first case 51 and the second case 52. As a result, the chamber space 53 is formed inside the housing 54, and this chamber space 53 is partitioned by the flexible membrane 55 into the first air chamber 53a and the second air chamber 53b. More specifically, the first air chamber 53a is formed so as to be surrounded by the inner face 62 of the first case 51 and the lower face of the flexible membrane 55, and the second air chamber 53b is formed so as to be surrounded by the inner face 72 of the second case 52 and the upper face of the flexible membrane 55.

In such a diaphragm chamber 50, the tube T2 is connected to the first air chamber port P5 on the front side. That is, as described above, in the configuration in FIG. 1, in each case of the pressure measurement units 5A to 5C, the tube T2 is the first line L21. Therefore, the other end of the first line L21 whose one end is connected to the second port P2 of the drip chamber 20 is connected to the first air chamber port P5 of the diaphragm chamber 50. As a result, the chamber space 21 of the drip chamber 20 and the first air chamber 53a of the diaphragm chamber 50 are in communication with each other via the first line L21.

Meanwhile, the first air chamber port P6 on the rear side is closed by a plug 90 having a columnar shape. After the second case 52 has been assembled to the first case 51, the plug is inserted and fixed. When two ports are formed in the first case in this manner, the first case 51 and the second case 52 can be assembled together without needing to take the orientation of the second case 52 with respect to the first case 51 into consideration, and thus, assemblability is improved. After the first case 51 and the second case 52 are assembled together, a plug, the first line, and the second line are fixed to the ports, as appropriate.

When the first air chamber port farther from the second air chamber port P7 is defined as the first air chamber port P5 in this manner, the opening 76a of the second air chamber port P7 can be prevented from being closed by the flexible membrane 55 pressed by the fluid (air) having flowed in from the first air chamber port P5. However, the positional relationship of the first air chamber ports P5, P6 with respect to the second air chamber port P7 is not limited to the above. For example, the first air chamber port nearer to the second air chamber port P7 may be defined as the first air chamber port P5, and the first air chamber port farther from the second air chamber port P7 may be defined as the first air chamber port P6. Alternatively, the first air chamber ports P5, P6 may be disposed at the same distance from the second air chamber port P7.

In the diaphragm chamber 50, the second air chamber port P7 has connected thereto one end of a second line L22 composed of a tube T5. A portion at the other end of this second line L22 is provided with a connector 40a, and the connector 40a allows connection to the connection port 40 (FIG. 1) of the pressure measurement device 4 of the dialysis device 2. Therefore, when the connector 40a is connected to the connection port 40, the second air chamber 53b of the diaphragm chamber 50 comes into communication with the pressure sensor S3 via the second line L22 and the first monitoring line L10 of the pressure measurement device 4. The inner diameter of the first line L21 is larger than the inner diameter of the second line L22. In the pressure measurement unit 5 and the diaphragm chamber 50 of the present disclosure, the first line L22 (tube T5) is not mandatory. That is, the connector 40a may be directly mounted to the second air chamber port P7 to omit the tube T5, thereby making it possible to connect the second air chamber port P7 to the connection port 40 only via the connector 40a.

The pressure measurement unit 5 described above is interposed at a point on the blood withdrawal line L1 or the blood reinfusion line L2 of the blood circuit 3 and connects the blood circuit 3 and the pressure measurement device 4 to each other, thereby enabling measurement of the pressure of the blood flowing in each line L1, L2. For example, with respect to the pressure measurement unit 5A provided to the blood withdrawal line L1, the blood taken from a patient flows in the blood withdrawal line LI to be dropped via the first port P1 into the chamber space 21 of the drip chamber 20A of the pressure measurement unit 5A. A predetermined volume of the blood is stored in the chamber space 21, whereby a liquid surface is formed. Therefore, the upper space in contact with the liquid surface of the blood in the chamber space 21 matches the pressure of the blood in the chamber space 21.

Meanwhile, the upper space of the chamber space 21 is in communication with the first air chamber 53a of the diaphragm chamber 50 via the first line L21. Therefore, the pressure in the first air chamber 53a matches (or becomes a pressure correlating with) the pressure of the blood, and the flexible membrane 55 deforms in accordance with this pressure. Due to this deformation of the flexible membrane 55, the pressure in the second air chamber 53b also matches (or becomes a pressure correlating with) the pressure of the blood, and the pressure (or the correlating pressure) of the blood propagates to the pressure sensor S3 via the second line L22. As a result, at the pressure sensor S3, measurement of the pressure of the blood flowing in the blood circuit 3 connected via the pressure measurement unit 5A is enabled. This also applies to the other pressure measurement units 5B, 5C.

Here, the pressure measurement unit 5 according to the present disclosure includes the drip chamber 20 and the diaphragm chamber 50. Thus, in addition to the pressure measurement described above, air evacuation and foreign matter removal by the drip chamber 20 are possible, and entry of the blood from the blood circuit 3 into the pressure measurement device 4 can be prevented by the flexible membrane 55 of the diaphragm chamber 50. Further, since the diaphragm chamber 50 is provided separately from the drip chamber 20, the possibility of generation of a clot can be reduced as compared with a case where a flexible membrane is provided in a drip chamber, and the shape and dimensions of each chamber 20, 50 can be optimized in accordance with the function thereof. For example, if the drip chamber 20 is downsized to reduce the dimension of the liquid surface of the blood (i.e., the opportunity for contact with air is reduced) and, at the same time, a large dimension (area) of the flexible membrane 55 is ensured to improve deformability, accurate pressure measurement can be realized.

As described above, with respect to the pressure measurement unit 5, the inner diameter of the first line L21 is larger than the inner diameter of the second line L22. Therefore, the pressure of the chamber space 21 of the drip chamber 20 can be more accurately transmitted to the first air chamber 53a of the diaphragm chamber 50, and entry of the blood from the chamber space 21 to the first air chamber 53a can be inhibited, whereby accurate pressure measurement can be realized.

In the pressure measurement unit 5, with respect to the diaphragm chamber 50, at least a part of the housing 54 thereof may be transparent so as to allow the inside thereof to be visually confirmed from outside, and at least a part of the flexible membrane 55 may have a chromatic color or be semi-transparent or opaque so as to be able to be visually confirmed, as a portion to be visually confirmed, through the housing 54. As an example, in the diaphragm chamber 50 according to the present disclosure, the entirety of the housing 54 is made of a transparent resin, and the entirety of the flexible membrane 55 exhibits a chromatic color. Thus, the state or movement of the flexible membrane 55 inside can be visually observed from outside of the diaphragm chamber 50, and thus, whether or not the state is a state where the pressure of the blood can be appropriately measured can be easily confirmed. The transparent portion of the housing 54 may exhibit a chromatic color or an achromatic color as long as the inside can be visually confirmed from outside. The position of the portion to be visually confirmed of the flexible membrane 55 only needs to be in a range where said portion can be visually confirmed from outside of the housing 54 through the transparent portion of the housing 54, and the degree of chromaticity of the color, semi-transparency, and opacity exhibited by the portion to be visually confirmed may be such that the presence can be recognized when viewed from outside through the transparent portion of the housing 54 and the sense of distance can be recognized.

The portion to be visually confirmed may have a combination of a plurality out of chromatic color, semi-transparency, or opacity, or may be a portion on which a pattern is formed using one or a plurality of chromatic color, semi-transparency, or opacity. The flexible membrane 55 can be configured by suitably using polyvinyl chloride or an elastomer (e.g., a styrene-based elastomer) among resins. As for the coloration of the portion to be visually confirmed of the flexible membrane 55, those of milky white (or milky translucent) can be suitably used. The optical physical property of the housing 54 and the flexible membrane 55 can also be specified by a haze value. For example, when a test piece having a thickness of 2 mm is measured according to JIS-K7136, the housing 54 preferably has a haze value not greater than 10, and at least the portion to be visually confirmed of the flexible membrane 55 has a haze value not less than 30 and not greater than 100, and preferably not less than 50 and not greater than 95, and those having a haze value of about 85, for example, can be adopted. From the viewpoint of visual confirmation, the flexible membrane 55 preferably has a chromatic color or is semi-transparent or opaque as described above, but not limited thereto, may be transparent.

In the diaphragm chamber 50 of the pressure measurement unit 5, the opening directions of the first air chamber port P5 and the second air chamber port P7 may cross each other. As an example, in the diaphragm chamber 50 according to the present disclosure, as described above, the first air chamber port P5 is open in the front-rear direction, and the second air chamber port P7 is open in the up-down direction. Thus, in a state where the pressure measurement unit 5 is set to the pressure measurement device 4, even if the direction in which the first line L21 extends and the direction in which the second line L22 extends cross each other, occurrence of a kink in the first line L21 or the second line L22 can be inhibited.

In the pressure measurement unit 5, at a point on the first line L21, the clamp C1 may be provided. Accordingly, when priming is performed before use of the pressure measurement unit 5, the saline that is used in priming can be prevented from entering the first air chamber 53a of the diaphragm chamber 50 through the first line L21.

In the diaphragm chamber 50 of the pressure measurement unit 5, in the inner face 62 of the case body 60 of the first case 51, the groove 65 forming a space that enables flowing of the fluid from the first air chamber port P5 to the first air chamber 53a through communication between the first air chamber 53a and the first air chamber port P5 is formed so as to be recessed with respect to the inner face 62. Thus, even when the flexible membrane 55 has largely been deflected to the first air chamber 53a side due to a negative pressure acting on the first air chamber 53a, the opening 66a of the first air chamber port P5 can be prevented from being closed by the flexible membrane 55.

On the inner face 72 of the case body 70 of the second case 52, the plurality of ribs 77 having a linear shape are provided in a protruding manner. These ribs 77 also prevent the opening 76a of the second air chamber port P7 from being closed by the flexible membrane 55. The configuration for preventing the opening 76a from being closed is not limited to the linear-shaped ribs 77, and may be a plurality of projections, or one or a plurality of grooves in communication with the second air chamber port P7.

When the maximum change amount of the volume of the first air chamber 53a caused by displacement of the flexible membrane 55 is defined as V1, and of the volume of the chamber space 21 of the drip chamber 20, the volume from a liquid level setting line 22L to the top face is defined as V2, the diaphragm chamber 50 of the present disclosure may satisfy 1.2×V1≤V2≤3.5×V1. More preferably, 1.5×V1≤V2≤3.2×V1 may be satisfied.

With respect to the diaphragm chamber 50, the larger the above V1 is, the larger the pressure measurement range becomes, but from the viewpoint of ease of handling the product on site, downsizing of the diaphragm chamber 50 is desired. Meanwhile, with respect to the pressure measurement unit 5 of the present disclosure, since the diaphragm chamber 50 is connected to the drip chamber 20, the space volume in which air is accommodated through communication between the chambers 20, 50 is large. Therefore, displacement of the flexible membrane 55 due to pulsation caused by operation of the liquid pump 11 is suppressed to a small level, and influence of the pulsation on pressure measurement can be suppressed. Therefore, the diaphragm chamber 50 can be downsized, and dimensions that satisfy the above formula can be realized. Accordingly, while high pressure measurement accuracy is ensured, downsizing of the diaphragm chamber 50 can be realized.

The “liquid level setting line 22L” of the drip chamber 20 relating to the volume V2 corresponds to the set level of the liquid surface that is positioned between the lower end position of the first port P1 and the upper end of the filter 24 and that is set in the designing stage of the drip chamber 20. In FIG. 3, a case where this liquid level setting line 22L is indicated by a line provided to the chamber body 22 is shown as an example, but the present disclosure is not limited thereto. For example, the liquid level setting line 22L may be indicated by a seal affixed to the outer face of the chamber body 22. Alternatively, the chamber body 20 may be configured as a joined structure of upper and lower two components and the liquid level setting line 22L may be indicated by the joining position. Still another display method may be adopted.

In the above description, as an example of the drip chamber 20, a vertical flow-in type has been described, but the present disclosure is not limited thereto. For example, a horizontal flow-in type drip chamber in which a liquid is introduced from the side face of the chamber body 22 to the chamber space 21 may be used. In the example in FIG. 1, a configuration in which the pressure measurement units 5 (5A to 5C) according to the present disclosure are provided at three points in the blood circuit 3 has been shown, but the present disclosure is not limited thereto. That is, at only some of these points, the pressure measurement unit 5 according to the present disclosure may be provided, or the pressure measurement unit 5 according to the present disclosure may be provided at another point. As for the other point, for example, the pressure measurement unit 5 may be provided so as to connect the collection line L6 and the pressure measurement device 4 to each other, and may be used to measure the pressure of the dialysate having passed through the filtration device 10 and flowing through the collection line L6.

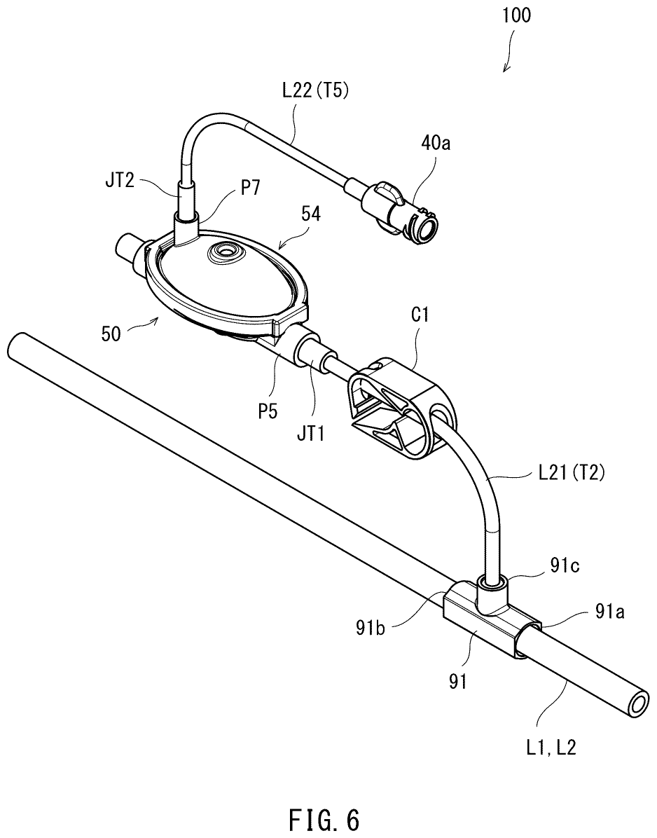

Meanwhile, the pressure measurement unit according to the present disclosure need not necessarily include a drip chamber, for the purpose of realizing accurate pressure measurement through prevention of entry of a liquid into the pressure measurement device 4, optimization of the dimensions of the flexible membrane 55, and the like. A pressure measurement unit 100 having such a configuration is shown in FIG. 6.

The pressure measurement unit 100 shown in FIG. 6 has a configuration in which the drip chamber 20 is omitted from the pressure measurement unit 5 shown in FIG. 2. That is, the pressure measurement unit 100 includes: the first line L21 having one end connected at a point on a line (e.g., the blood withdrawal line L1 or the blood reinfusion line L2) of the blood circuit 3; the clamp C1 provided at a point on the first line L21; the diaphragm chamber 50 in which the other end of the first line L21 is connected to the first air chamber port P5; and the second line L22 having one end connected to the second air chamber port P7 of the diaphragm chamber 50 and the other end connected to the connection port 40 of the pressure measurement device 4. The configurations of these elements are the same as the corresponding elements of the pressure measurement unit 5 having been already described.

Since the pressure measurement unit 100 having such a configuration includes the diaphragm chamber 50 having the flexible membrane 55, the pressure measurement unit 100 can prevent entry of the blood into the pressure measurement device 4 and can realize accurate pressure measurement through optimization of the dimensions of the flexible membrane 55 as described above. Further, the first air chamber port P5 and the second air chamber port P7 cross each other in the opening direction. Therefore, similar to the pressure measurement unit 5, occurrence of a kink in the first line L21 or the second line L22 can be inhibited.

A joint tube formed from a material more flexible than that of the housing 54 may be provided before or after the housing 54 of the diaphragm chamber 50. That is, to the first air chamber port P5 of the diaphragm chamber 50, the first line L21 (tube T2) may be directly connected, but may be connected via a joint tube JT1 having a cylindrical shape (FIG. 2, FIG. 4, FIG. 5A, FIG. 6). For example, as shown in FIG. 5A, the first air chamber port P5 has a round tubular shape, and inside thereof, has a port space 68a having a large diameter and having a cylindrical shape, and a port hole 68b having a small diameter and positioned on the depth side (rear side) of the port space 68a to be in communication with the first air chamber 53a. The port space 68a and the port hole 68b are connected to each other via a step portion 68c.

The joint tube JT1 is inserted in the port space 68a of the first air chamber port P5, and further, an end portion of the tube T2 is inserted in this joint tube TJ1. More specifically, the joint tube JT1 is provided such that the outer peripheral face of a rear portion thereof (approximately, the rear half in the longitudinal direction) is in contact with the inner peripheral face defining the port space 68a of the first air chamber port P5 and such that the rear end face is in contact with the step portion 68c, and the faces in contact with each other are connected with an adhesive or the like. The front portion (approximately, the front half in the longitudinal direction) of the joint tube JT1 protrudes (extends) outward (frontward) from the first air chamber port P5.

A rear end portion of the tube T2 is inwardly inserted through the front opening of the joint tube JT1, and the rear end of the tube T2 is positioned on the front side with respect to the rear end of the joint tube JT1 and is at a position near the center in the longitudinal direction of the joint tube JT1, for example. Such a joint tube JT1 is formed from a material more flexible than that of the housing 54, and can be composed of the same polyvinyl chloride resin as that of the tube T2, for example. The joint tube TJ1 and the tube T2 may be formed from different materials.

Similarly, to the second air chamber port P7 of the diaphragm chamber 50, the second line L22 (tube T5) may be directly connected, but may be connected via a joint tube JT2 having a cylindrical shape (FIG. 2, FIG. 4, FIG. 5A, FIG. 6). For example, as shown in FIG. 5A, the second air chamber port P7 has a round tubular shape, and inside thereof, has a port space 78a having a large diameter and having a cylindrical shape, and a port hole 78b having a small diameter and positioned on the depth side (rear side) of the port space 78a to be in communication with the second air chamber 53b. The port space 78a and the port hole 78b are connected to each other via a step portion 78c.

The connection manner of the second air chamber port P7, the joint tube JT2, and the tube T5 is the same as the connection manner of the first air chamber port P5, the joint tube JT1, and the tube T2 described above, and thus, description thereof is omitted here. The lower end of the tube T5 is positioned on the upper side with respect to the lower end of the joint tube JT2, and is at a position near the center in the longitudinal direction of the joint tube JT2, for example. The joint tube JT2 is formed from a material more flexible than that of the housing 54, and can be composed of the same polyvinyl chloride resin as that of the tube T5, for example. The joint tube JT2 and the tube T5 may be formed from different materials.

As described above, since the tube T2 is inserted in the joint tube JT1 extending outward of the first air chamber port P5, occurrence of kinking of the tube T2 (i.e., bending such that the internal space is closed) in the vicinity of the first air chamber port P5 can be prevented. With respect to the tube T5 connected to the second air chamber port P7, similarly, with the interposition of the joint tube JT2, occurrence of a kink of the tube T5 is prevented. When the tube T5 has a smaller inner diameter than the tube T2, it is possible to prevent a solvent or the like from closing the flow path of the tube T5. That is, in a case where the tube T5 has a small diameter, when a bonding agent such as a solvent or the like is attached, the bonding agent may flow inside due to capillary phenomenon. In a case where the joint tube JT2 is interposed, after the tube T5 and the joint tube JT2 are connected, it is possible to blow in air to expel the bonding agent outside the tube T5. As described above, in the present disclosure, the lower end of the tube T5 is positioned on the upper side with respect to the lower end of the joint tube JT2. Therefore, the bonding agent connecting the tube T5 and the joint tube JT2 can be inhibited from flowing into the small-diameter tube T5 due to capillary phenomenon.

Such a joint tube may be provided at the connection point between the first line L21 and a three-way joint 91 provided at a point on the blood circuit 3 (the blood withdrawal line L1, the blood reinfusion line L2, or the like), as shown in FIG. 6. That is, out of the three connecting ports of the three-way joint 91, the line of the blood circuit 3 is connected to two connecting ports 91a, 91b and the first line L21 (tube T2) is connected to the remaining one connecting port 91c. At the connection of the tube T2 to this connecting port 91c, the joint tube may be interposed.

Further, such a joint tube may be provided at the connection point between the first line L21 and the second port (pressure measurement port) P2 of the drip chamber 20 shown in FIG. 2.

The connection manner in this case is the same as the connection manner of the first air chamber port P5, the joint tube JT1, and the tube T2 described above, and thus, description thereof is omitted here. The joint tube used in this case is formed from a material more flexible than that of the three-way joint 91, and can be composed of the same polyvinyl chloride resin as that of the tube T2, for example. The joint tube and the tube T2 may be formed from different materials. With this connection manner, occurrence of kinking of the tube T2 in the vicinity of the three-way joint 91 can be prevented.

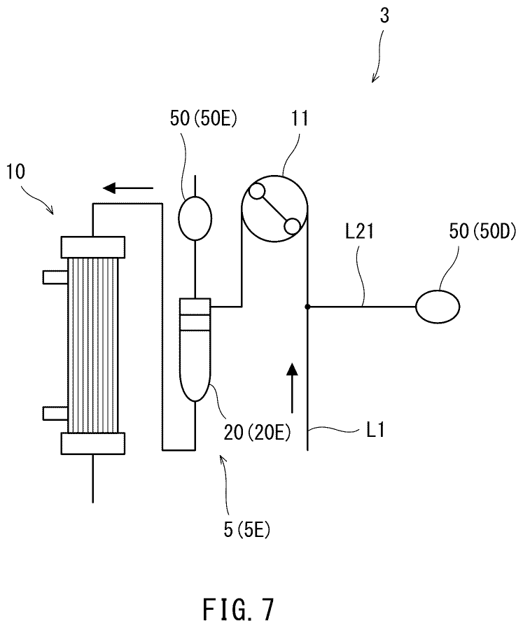

Meanwhile, in order to inhibit flowing of the blood into the diaphragm chamber 50 provided by being branched from the blood withdrawal line L1 or the blood reinfusion line L2, it is possible to adopt the following configuration for the blood circuit 3. The configuration of this blood circuit 3 will be described with reference to the schematic diagram in FIG. 7.

The blood circuit 3 (only a part thereof is shown) shown in FIG. 7 has the blood withdrawal line (blood line) L1 that connects the blood vessel of the patient and the filtration device 10 to each other. The blood pump 11 is provided at a point on this blood withdrawal line L1. More specifically, the blood pump 11 is composed of a roller pump, and the portion where the blood pump 11 is provided on the blood withdrawal line L1 forms a pumping tube. That is, a pumping tube forming a part of the blood withdrawal line L1 is fixed to the blood pump 11. On the blood withdrawal line L1, between the blood pump 11 and the filtration device 10, a pressure measurement unit 5 (5E) composed of a drip chamber 20 (20E) and a diaphragm chamber 50 (50E) is provided.

At a position on the upstream side of the blood pump 11 on the blood withdrawal line L1, one end of the first line L21 forming a branch line is connected. To the other end of this first line L21, a diaphragm chamber 50 (50D) is connected. In the same manner as described above, the diaphragm chamber 50D has: the first air chamber 53a in communication with the first line L21; the flexible membrane 55 in contact with the first air chamber 53a; and the second air chamber 53b separated from the first air chamber 53a by the flexible membrane 55 (see FIG. 5A). Here, the blood withdrawal line L1 and the first line L21 are connected via a three-way joint as in the configuration in FIG. 6.

Here, the roller pump moves the gripping position of the pumping tube, thereby moving the liquid in the tube. Therefore, to the blood in the blood withdrawal line L1, a positive pressure is applied on the downstream side of the roller pump, and a negative pressure is applied on the upstream side of the roller pump. Then, in the blood circuit 3 in FIG. 7, the first line L21, which is a branch line in communication with the diaphragm chamber 50D, is positioned on the upstream side of the blood pump 11 composed of the roller pump, on the blood withdrawal line L1. Therefore, a negative pressure is also generated in the first line L21, air in the first line L21 moves to the blood withdrawal line L1 side, and in exchange for this air, the blood in the blood withdrawal line L1 can flow into the first line L21.

In this regard, in the blood circuit 3 shown in FIG. 7, the length of the first line L21, which is a branch line, is set to be not less than 100 mm. Therefore, even if the blood has flowed into the first line L21 from the blood withdrawal line L1 due to the action of the negative pressure, it is possible to inhibit the blood having flowed from reaching the diaphragm chamber 50D. When routability of the branch line is taken into consideration, the length of the first line L21 is preferably not less than 100 mm and not greater than 500 mm, more preferably not less than 100 mm and not greater than 300 mm, and further preferably not less than 150 mm and not greater than 200 mm.

### INDUSTRIAL APPLICABILITY

The present disclosure can be suitably applied to a pressure measurement unit provided between a liquid circuit and a pressure measurement device.

### REFERENCE CHARACTER LIST

3 blood circuit (liquid circuit)
4 pressure measurement device
5 pressure measurement unit
20 drip chamber
21 chamber space
50 diaphragm chamber
53a first air chamber
53b second air chamber
54 housing
55 flexible membrane
65 groove
C1 clamp
L21 first line
L22 second line
P1 first port (inlet port)
P2 second port (pressure measurement port)
P4 fourth port (outlet port)
P5 first air chamber port
P7 second air chamber port

1. A pressure measurement unit provided between a liquid circuit and a pressure measurement device, the pressure measurement unit comprising:
a drip chamber having a chamber space, an inlet port configured to introduce a liquid flowing in the liquid circuit into the chamber space, an outlet port configured to send out the liquid in the chamber space to the liquid circuit, and a pressure measurement port in communication with an upper portion of the chamber space;
a first line having a tubular shape and having one end connected to the pressure measurement port; and
a diaphragm chamber having a first air chamber in communication with another end of the first line, a flexible membrane in contact with the first air chamber, and a second air chamber separated from the first air chamber by the flexible membrane.

2. The pressure measurement unit according to claim 1, further comprising
a second line having a tubular shape and having one end connected to the second air chamber and another end connected to the pressure measurement device, wherein
an inner diameter of the first line is larger than an inner diameter of the second line.

3. The pressure measurement unit according to claim 1, wherein
the diaphragm chamber has a housing accommodating the first air chamber, the second air chamber, and the flexible membrane, and
at least a part of the housing is transparent so as to allow an inside thereof to be visually confirmed from outside, and at least a part of the flexible membrane has a chromatic color or is semi-transparent or opaque.

4. The pressure measurement unit according to claim 1, wherein
the diaphragm chamber has a first air chamber port in communication with the first air chamber, and a direction of deformation of the flexible membrane due to differential pressure change between the first air chamber and the second air chamber and a flowing direction of a fluid in the first air chamber port cross each other.

5. The pressure measurement unit according to claim 1, wherein
the diaphragm chamber has a housing accommodating the first air chamber, the second air chamber, and the flexible membrane, and a first air chamber port formed at the housing and configured to allow communication between the first air chamber and the first line, and
the first air chamber is defined by an inner face of the housing and the flexible membrane, and at the inner face of the housing, a recess or a protrusion forming a space that allows communication between the first air chamber and the first air chamber port is formed.

6. The pressure measurement unit according to claim 5, wherein
the space extends in a direction away from the first air chamber port.

7. The pressure measurement unit according to claim 1, wherein
when a maximum change amount of a volume of the first air chamber caused by displacement of the flexible membrane in the diaphragm chamber is defined as V1, and of a volume of the chamber space of the drip chamber, a volume from a liquid level setting line to a top face is defined as V2, 1.2×V1≤V2≤3.5×V1 is satisfied.

8. The pressure measurement unit according to claim 1, wherein
between the first line and a housing of the diaphragm chamber, a joint tube that is more flexible than the housing of the diaphragm chamber is provided.

9. A pressure measurement unit provided between a liquid circuit and a pressure measurement device, the pressure measurement unit comprising:
a first line having a tubular shape and branched from the liquid circuit; and
a diaphragm chamber having a first air chamber in communication with the first line, a flexible membrane in contact with the first air chamber, and a second air chamber separated from the first air chamber by the flexible membrane, wherein
between the first line and a housing of the diaphragm chamber, a joint tube that is more flexible than the housing of the diaphragm chamber is provided.

10. A diaphragm chamber, for a pressure measurement unit, provided between a liquid circuit and a pressure measurement device and connected to a first line branched from the liquid circuit, the diaphragm chamber comprising:
a first air chamber in communication with the first line; a flexible membrane in contact with the first air chamber; a second air chamber separated from the first air chamber by the flexible membrane; and a first air chamber port in communication with the first air chamber, wherein
a direction of deformation of the flexible membrane due to differential pressure change between the first air chamber and the second air chamber and a flowing direction of a fluid in the first air chamber port cross each other.

11. A blood circuit serving as the liquid circuit and having a pumping tube fixed to a roller pump at a point on a blood line, the blood circuit comprising:
a branch line serving as the first line and having one end connected at a position on an upstream side of the pumping tube on the blood line; and
the diaphragm chamber according to claim 10 connected to another end of the branch line, wherein
a length of the branch line is not less than 100 mm.
